(19) United States
(12) Patent Application Publication
(10) Pub. No.: US 2015/0119339 A1
(43) Pub. Date: Apr. 30, 2015

(54) USE OF HMB TO IMPROVE HEALTH OUTCOMES FOR HOSPITALIZED PATIENTS

(71) Applicant: ABBOTT LABORATORIES, ABBOTT PARK, IL (US)

(72) Inventors: Menghua Luo, New Albany, OH (US); Jeffrey L. Nelson, Dublin, OH (US); Anne C. Voss, Columbus, OH (US); Owen J. Kelly, Pataskala, OH (US); Geraldine E. Baggs, Columbus, OH (US); Min Tian, New Albany, OH (US); Jamie Partridge, Columbus, OH (US)

(73) Assignee: ABBOTT LABORATORIES, ABBOTT PARK, IL (US)

(21) Appl. No.: 14/406,886

(22) PCT Filed: Jun. 10, 2013
(86) PCT No.: PCT/US2013/044899

§ 371 Date: Dec. 10, 2014

Related U.S. Application Data
(60) Provisional application No. 61/658,078, filed on Jun. 11, 2012.

(51) Int. Cl. A23L 1/305; A61K 31/19; A23L 1/29

(52) U.S. Cl. CPC A23L 1/3051; A23L 1/293; A23L 1/296; A61K 31/19; A23V 2002/00
USPC 514/21.2; 426/2; 514/557

(57) ABSTRACT

A method of care is described that includes administering an effective amount of β-hydroxy-β-methylbutyrate (HMB) to an adult or elderly patient to decrease the risk of an adverse health event during or subsequent to hospitalization of the patient.

### RELATED APPLICATIONS

This application claims priority to and the benefit of U.S. Provisional Patent Application Ser. No. 61/658,078, entitled “USE OF HMB TO IMPROVE HEALTH OUTCOMES FOR HOSPITALIZED PATIENTS” and filed Jun. 11, 2012, the entire disclosure of which is incorporated herein by reference.

### BACKGROUND

The elderly (65 years of age or older) account for 38% of hospital stays and utilize close to 50% of total hospital expenditures in the US. Acute illness-related short-stay hospitalization in the elderly is frequently associated with a loss of lean body mass (LBM) and function, which can lead to loss of independence, a need for post-discharge assistance, long-term institutionalization and caregiver burden.

Regardless of the type of acute illness, the prolonged period of immobility or bed rest alone has a pronounced effect on rapid muscle atrophy, which is primarily due to decreased protein synthesis and/or increased protein degradation. With illness or injury, there is a greater extent of LBM loss. In three separate studies, the effect of bed rest on LBM loss was assessed in healthy young volunteers, healthy elderly volunteers, and elderly inpatients. Healthy young volunteers lost 2% (˜500 g=1.1b) of LBM in the leg during 28 days of bed rest, healthy elderly subjects lost 10% (˜1000 g=2.2 lb) of leg LBM during 10 days of bed rest, while elderly inpatients lost greater than 10% of leg LBM within only 3 days of hospitalization. Functional impairment occurs with LBM loss, 65% of elderly patients experience a decline in mobility with hospitalization, and 35% report decreased activities of daily living (ADL).

Other deleterious factors associated with hospitalization, such as physiological stress and malnutrition may result in greater extent of loss in muscle mass and function. It has been estimated that 25-60% of geriatric patients in hospitals and nursing homes have shown evidence of malnutrition. In the acute care hospital setting, around 21% of patients have an average daily in-hospital nutrient intake of less than 50% of their calculated maintenance energy requirements.

Analysis of Medicare claim data from 2003-2004 revealed that 19.6% of the patients were rehospitalized within 30 days post discharge, and 34.0% within 90 days. Among the more common reasons reported for rehospitalization were heart failure, acute myocardial infarction, chronic obstructive pulmonary disease, pneumonia and hip fracture. Measures of poor health such as comorbidity burden, prior medical services use, and increasing age were associated with re-admission. Few re-admission risk models have been identified for three of the most common reasons for rehospitalization such as heart failure, chronic obstructive pulmonary disease and myocardial infarction. Similarly, the Agency for Healthcare Research and Quality reported a 19% 30-day re-admission rate among the Medicare patients age 65 or older in 2008, based on data from 15 states, and rehospitalizations in Medicare were estimated to cost $17 billion a year. Re-admission within 30 days post-discharge has been considered a marker of low quality care, and Congress has passed the Patient Protection and Affordable Care Act (PPACA), which gives Centers for Medicare and Medicaid Services (CMS) the authority to penalize hospitals for excess re-admission rates starting the federal fiscal year.

Re-admission risk prediction remains a poorly understood and complex endeavor. Models of patient-level factors such as medical co-morbidities, basic demographic data and clinical variables are much better able to predict mortality than re-admission. Broader social, environmental, and medical factors such as access to care, social support, substance abuse, and functional status contribute to re-admission risk in some models, but the utility of such factors has not been widely studied. It is likely that hospital and health system-level factors, which are not present in current re-admission risk models, contribute to risk. For instance, the timeliness of post-discharge follow-up, coordination of care with the primary care physician, and quality of medication reconciliation may be associated with re-admission risk. The supply of hospital beds may independently contribute to higher re-admission rates. Finally, the quality of inpatient care could contribute to risk although the evidence is mixed. Public reporting and financial penalties for hospitals with high 30-day re-admission rates are spurring organizations to innovate and implement quality improvement programs. Use of re-admission rates as a quality indicator assumes that re-admissions are related to poor quality care and are potentially preventable. However the cause and preventability of re-admissions remains unclear and understudied.

### SUMMARY

In one aspect, a method of care is provided that includes administering an effective amount of β-hydroxy-β-methylbutyrate (HMB) to an adult or elderly patient to decrease the risk of an adverse health event during or subsequent to hospitalization of the patient.

In another aspect, a method of care is provided that includes administering from about 1 to 10 g/day of β-hydroxy-β-methylbutyrate (HMB) in a liquid nutritional supplement at least once daily to an elderly patient to decrease the risk of an adverse health event during and up to about 1 year after hospitalization of the patient.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

### DEFINITIONS

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The term “adult,” as used herein, refers to an individual of at least 21 years of age.

The term “elderly,” as used herein, refers to an individual of at least 45 years of age, including at least 50 years of age, at least 55 years of age, at least 60 years of age, at least 65 years of age, at least 70 years of age, at least 75 years of age, and including at least 80 years of age or greater. The term “elderly” also includes the groups of from about 45 years of age to about 95 years of age, and the group of from about 55 years of age to about 80 years of age.

The term “patient,” as used herein, refers to a male or female human who is being treated for a medical condition.

The term “hospital,” as used herein, is a health care institution providing patient treatment by specialized staff (e.g., physicians, surgeons, nurses, and dieticians) and equipment. Hospitals include general hospitals, which are set up to deal with many kinds of disease and injury, and typically have an emergency department to deal with immediate and urgent threats to health, as well as specialized hospitals. Specialized hospitals include trauma centers, rehabilitation hospitals, children's hospitals, seniors' (geriatric) hospitals, teaching hospitals, and hospitals for dealing with specific medical needs such as psychiatric problems and certain disease categories such as cardiac, oncology, or orthopedic problems. A hospital includes bedding and other facilities required for inpatient care, as opposed to a health care institution such as a clinic that only provides for outpatient care. Hospitalization refers to the act of a patient being entered or staying within a hospital.

A method of care is described that includes administering an effective amount of β-hydroxy-β-methylbutyrate (HMB) to an adult or elderly patient to decrease the risk of an adverse health event during or subsequent to hospitalization of the patient. While HMB will typically provide the most benefit for elderly patients, it can also be helpful for treating adult patients, in particular those whose health has been compromised as a result of disease, treatment side-effects, or prolonged bed rest.

β-hydroxy-β-methylbutyrate (HMB) is a metabolite of the essential amino acid leucine, and has the IUPAC name 3-hydroxy-3-methylbutanoic acid. A preferred form of HMB is the calcium salt of HMB, also designated as Ca-HMB, which is most typically the monohydrate calcium salt. The HMB used can come from any source. Calcium HMB monohydrate is commercially available from Technical Sourcing International (TSI) of Salt Lake City, Utah. Note that all of the amounts of HMB described herein are based on use of Ca-HMB.

Although calcium monohydrate is the preferred form of HMB for use herein, other suitable sources include HMB as a free acid, a salt, an anhydrous salt, an ester, a lactone, or other product forms that provide a bioavailable form of HMB suitable for administration. Non-limiting examples of suitable salts of HMB for use herein include HMB salts, hydrated or anhydrous, of sodium, potassium, chromium, calcium, or other non-toxic salt forms.

β-hydroxy-β-methylbutyrate (HMB) is administered to decrease the risk of an adverse health event. The risk of an event, as defined herein, refers to the likelihood that a patient who is being treated according to the present method will suffer an adverse health event. The baseline level of risk can be determined based on existing statistics regarding patients who are not being administered HMB. A difference in the risk (e.g., a decrease) represents a change in the probability that a given patient will suffer an adverse health event. This change in risk can be determined through studies of a group of patients being administered HMB using a statistically relevant number of patients, as described in the Example herein. Risk represents a probability, and can be assigned to an individual on a percentage basis, though of course risk does not imply certainty that a specific event such as an adverse health event will occur or not occur.

The term “an effective amount” is intended to qualify the amount of HMB which will achieve the goal of decreasing the risk that the patient will suffer an adverse health event, while avoiding adverse side effects such as those typically associated with alternative therapies. The effective amount may be administered in one or more doses.

HMB is administered to decrease the risk of an adverse health event during or subsequent to hospitalization of the patient. An adverse health event (AHE) is any untoward medical occurrence in a patient. An AHE can therefore be any unfavorable and unintended sign (including abnormal laboratory findings deemed to be clinically significant), symptoms, or disease arising in the patient. Overall, an objective of HMB administration as described herein is to decrease the health care cost resulting from care of a patient.

Health care costs can arise from a wide variety of factors, such as, for example, the need for rehospitalization or extended hospitalization, the need for additional drugs or procedures, or any form of additional care by medical health care professionals such as a doctor's appointment or home care visit. Extended hospitalization, i.e., an increase in the duration of hospitalization in comparison to the initially expected duration of the hospitalization, can include an increase by 10%, an increase by 25%, an increase by 50%, and an increase by 100% or more relative to the initially expected duration of hospitalization. The additional health care costs can arise during the initial hospitalization, or can be a result of post-discharge medical care. Examples of post-discharge medical care include a visit to a health practitioner, a home visit by a health practitioner, entry into a nursing home, or rehabilitation therapy.

An adverse health event can require rehospitalization of a patient. Typically this would be the case if the adverse health care event takes place after the patient has been released from an initial hospitalization. Hence, this return to the hospital by a patient after the initial period of hospitalization is referred to herein as “rehospitalization.” The rehospitalization can occur at any time subsequent to the initial hospitalization. However, rehospitalization occurring closer in time to the initial hospitalization is more likely to be correlated with health issues that existed or developed during the initial hospitalization. Accordingly, in different embodiments, the rehospitalization can occur within about 1 month, within about 3 months, within about 6 months, within about 9 months, or within about 1 year of the initial hospitalization.

Adverse health events can include a wide variety of different events, and can have a variety of levels of severity. For example, death or incapacity or a health event that is life threatening are all examples of severe adverse health events. Other examples of serious adverse health events include events requiring inpatient hospitalization or prolongation of existing hospitalization. More specifically, an adverse health event can include an injurious fall; a respiratory infection; pneumonia; a urinary tract infection; influenza; pressure ulcer; acute cardiac events, including cardiac ischemia, cardiac failure, cardiac arrest, etc.; and thrombosis. Other adverse health events include a significant decline in mental health (e.g., cognition) or a significant decrease in body mass, in particular lean body mass.

Any worsening of a pre-existing condition or illness is considered an AHE. An elective surgery/procedure scheduled to occur after hospitalization of a patient will not be considered an AHE if the surgery/procedure is being performed for a pre-existing condition and was pre-planned prior to hospitalization of the patient. However, if the pre-existing condition deteriorates unexpectedly during or subsequent to hospitalization (e.g. surgery has to be performed earlier than planned), then the deterioration of the condition for which the elective surgery/procedure is being done will be considered an AHE.

Malnourishment and sarcopenia are conditions frequently seen in hospitalized elderly patients. Accordingly, in some embodiments, the patient being administered an effective amount of HMB is malnourished immediately prior to administration of the HMB. A patient who is malnourished is a patient whose nutrient intake cannot meet increased demand due to illness or other conditions or a patient who has been receiving an unbalanced diet in which certain nutrients are lacking, in excess (too high an intake), or in the wrong proportions. Malnourishment can be readily identified by those skilled in the art. Patients who are malnourished can be expected to benefit particularly from HMB that is provided in a nutritional composition.

In another embodiment, the patient being administered an effective amount of HMB has sarcopenia. Sarcopenia is a degenerative loss of skeletal muscle mass and strength that is generally associated with aging, but can also be caused by a significant loss of opportunity for movement, as can occur as a result of immobility due to prolonged bed rest. Sarcopenia differs from regular muscle atrophy, and involves a loss of lean body mass and replacement of that body mass with fat. While sarcopenia is generally seen as one of the effects of aging, it can exist to substantially different degrees in individuals based on genetic and environmental factors, such as the extent to which an individual exercises. In further embodiments, the patient may be suffering from severe sarcopenia, which is an extreme form of sarcopenia which can have a major effect on the mobility and vigor of a patient. Simple circumference measurement does not provide enough data to determine whether or not an individual is suffering from severe sarcopenia. Sarcopenia is also marked by a decrease in the circumference of distinct types of muscle fibers. Skeletal muscle has different fiber-types, which are characterized by expression of distinct myosin variants. During sarcopenia, there is a decrease in “type 2” fiber circumference (Type II), with little to no decrease in “type 1” fiber circumference (Type I).

The HMB can be formulated in suitable compositions and then, in accordance with the methods of the invention, administered to a patient in a form adapted to the chosen route of administration. The formulations include, but are not limited to, those suitable for oral or non-oral (including subcutaneous, intramuscular, intraperitoneal, intratumoral, and intravenous) administration. Oral administration, as defined herein, includes any form of administration in which the HMB passes through the esophagus of the patient. For example, oral administration includes nasogastric intubation, in which a tube is run from through the nose to the stomach of the patient to administer food or drugs.

Oral formulations include any solid, liquid, or powder formulation suitable for use herein, provided that such a formulation allows for the safe and effective oral delivery of HMB and optional nutritive components. In preferred embodiments, the oral formulation is a liquid nutritional composition. Formulations of the present invention suitable for oral administration may be presented as discrete units such as tablets, troches, capsules, lozenges, wafers, or cachets, each containing a predetermined amount of the HMB as a powder or granules or as a solution or suspension in an aqueous liquor or non-aqueous liquid such as a syrup, an elixir, an emulsion, or a draught.

The concentration of HMB in the nutritional composition may range up to about 10%, including from about 0.01% to about 10%, and also including from about 0.1% to about 5.0% and also including from about 0.5% to about 2%, and also including from about 0.4% to about 1.5% by weight of the nutritional liquid composition.

The nutritional composition administered to the patients can provide from about 0.1 g/day to about 10 g/day of HMB. Alternately, the composition can provide about 0.5 g/day to about 10 g/day of HMB, about 1.0 g/day to about 10 g/day, or about 0.5 g/day to about 5 g/day. Patients may be administered one serving per day, two servings per day, three servings per day, or four or more servings per day to receive the desired amount of HMB from the nutritional composition.

The HMB can be administered during and/or subsequent to hospitalization of the patient. For example, the HMB can be administered only during the initial hospitalization of the patient, or only during a portion of the initial hospitalization of the patient. Alternately, or in addition, the HMB can be administered subsequent to hospitalization of the patient. Administration subsequent to hospitalization includes administration of HMB to the patient beginning immediately or shortly after release from initial hospitalization. For example, administration subsequent to hospitalization can begin up to one week after release, 3 days after release, 1 day after release, or immediately after release. Administration after release can also continue for as long as it is useful for the patient to be receiving HMB. For example, administration can continue for one month, three months, 6 months, 9 months, or a year or more after release from initial hospitalization.

In a preferred embodiment, the HMB is administered to the patient as part of a nutritional composition. The nutritional composition includes one or more ingredients that help satisfy the patients nutritional requirements, in addition to providing a useful formulation for the HMB. For example, the nutritional composition can include a fat, a carbohydrate, and/or a protein. Preferably, the nutritional composition includes at least one source of fat, at least one source of carbohydrate, and at least one source of protein. In some embodiments, the nutritional composition can be formulated to provide a specialized nutritional product for use in patients afflicted with specific diseases or conditions. Many different sources and types of proteins, lipids, and carbohydrates are known and can be used in nutritional compositions including HMB. Preferably, the nutritional composition is in the form of a powder suitable for reconstitution to a liquid, a liquid or a bar.

Examples of nutritional composition forms suitable for use herein include snack and meal replacement products, including those formulated as bars, sticks, cookies or breads or cakes or other baked goods, frozen liquids, candy, breakfast cereals, powders or granulated solids or other particulates, snack chips or bites, frozen or retorted entrees, and so forth. The nutritional composition can also be in a form that fall between solid and liquid, such as semi-solid or semi-liquid compositions such as puddings or gels.

Examples of liquid product forms suitable for use herein include snack and meal replacement products, hot or cold beverages, carbonated or non-carbonated beverages, juices or other acidified beverages, milk or soy-based beverages, shakes, coffees, teas, compositions for administration by nasogastric intubation, and so forth. These liquid compositions are most typical formulated as suspensions or emulsions, but can also be formulated in any other suitable form such as clear liquids, substantially clear liquids, liquid gels, and so forth.

The nutritional composition includes one or more ingredients that help satisfy the patients' nutritional requirements. The optional nutrients can provide up to about 1000 kcal of energy per serving or dose, including from about 25 to about 900 kcal, from about 75 to about 700 kcal, from about 150 to about 500 kcal, from about 350 to about 500 kcal, or from about 200 to about 300 kcal.

Carbohydrates suitable for use in the composition may be simple, complex, or variations or combinations thereof. Non-limiting examples of suitable carbohydrates include hydrolyzed or modified starch or cornstarch, maltodextrin, glucose polymers, sucrose, corn syrup, corn syrup solids, rice-derived carbohydrate, glucose, fructose, lactone, high fructose corn syrup, indigestible oligosaccharides (e.g., fructooligosaccharides), soluble or insoluble fiber, honey, sugar alcohols (e.g., maltitol, erythritol, sorbitol) and combinations thereof.

Proteins suitable for use in the compositions, in addition to the HMB components described herein, include hydrolyzed, partially hydrolyzed or non-hydrolyzed proteins or protein sources, and can be derived from any known or otherwise suitable source such as milk (e.g., casein, whey), animal (e.g., meat, fish), cereal (e.g., rice, corn), vegetable (e.g., soy), or combinations thereof. In some embodiments, the nutritional composition can include a high amount of protein. For example, the nutritional composition can include at least 10 grams of protein.

Fats suitable for use in the compositions include coconut oil, fractionated coconut oil, soy oil, corn oil, olive oil, safflower oil, high oleic safflower oil, MCT oil (medium chain triglycerides), sunflower oil, high oleic sunflower oil, palm and palm kernel oils, palm olein, canola oil, marine oils, cottonseed oils, and combinations thereof.

The HMB-containing nutritional composition can also include other ingredients that may modify the physical, nutritional, chemical, hedonic, or processing characteristics of the product or serve as pharmaceutical or additional nutritional components. Non-limiting examples of such optional ingredients include preservatives, antioxidants, emulsifying agents, buffers, fructooligosaccharides, chromium picolinate, pharmaceutical additives, colorants, flavors or masking agents, thickening agents and stabilizers, artificial sweeteners, hydrocolloids such as guar gum, xanthan gum, carrageenan, gellan gum, gum acacia, and so forth.

The HMB-containing nutritional composition can also include vitamins or related nutrients, examples of which include vitamin A, vitamin D, vitamin E, vitamin K, thiamine, riboflavin, pyridoxine, vitamin B12, carotenoids, niacin, folic acid, pantothenic acid, biotin, vitamin C, choline, inositol, and salts and combinations thereof.

Examples of preferred nutritional compositions are described in Tables 1A and 1B below, with the specific ingredients provided immediately thereafter.

TABLE 1A

| | | Vanilla or | |
|---|---|---|---|
| | | Strawberry | |
| | | Flavored | Chocolate Flavored |
| | | Nutritional | Nutritional |
| | | Composition | Composition |
| | UNIT | per 8 fl oz | per 8 fl oz |
| Energy EU | kcal | 350 | 350 |
| Protein | g | 20 | 20 |
| Fat | g | 11 | 11 |
| Linoleic acid | g | 3 | 3 |
| Carbohydrate | g | 44 | 45 |
| Fructooligosaccharide | g | 3 | 2 |
| Sugar | g | 20 | 20 |
| Ca-HMB | g | 1.5 | 1.5 |
| VITAMINS | | | |
| Vitamin A (Palmitate) | IU | 1000 | 1000 |
| Vitamin A (B- | IU | 0 | 0 |
| Carotene) | | | |
| Vitamin D₃ | IU | 160 | 160 |
| Vitamin E | IU | 30 | 30 |
| Vitamin K₁ | mcg | 20 | 20 |
| Vitamin C | mg | 60 | 60 |
| Folic Acid | mcg | 200 | 200 |
| Vitamin B₁ | mg | 0.38 | 0.38 |
| Vitamin B₂ | mg | 0.43 | 0.43 |
| Vitamin B₆ | mg | 0.5 | 0.5 |
| Vitamin B₁₂ | mcg | 3 | 3 |
| Niacin | mg | 5 | 5 |
| Pantothenate | mg | 2.5 | 2.5 |
| Biotin | mcg | 75 | 75 |
| L-carnitine | mg | 43 | 43 |
| Choline | mg | 83 | 83 |
| MINERALS | | | |
| Sodium | mg | 240 | 240 |
| Potassium | mg | 560 | 630 |
| Chloride | mg | 150 | 150 |
| Calcium | mg | 500 | 500 |
| Phosphorus | mg | 350 | 350 |
| Magnesium | mg | 100 | 100 |
| Iron | mg | 4.5 | 4.5 |
| Zinc | mg | 15 | 15 |
| Manganese | mg | 0.50 | 0.70 |
| Copper | mg | 0.50 | 0.75 |
| Iodine | mcg | 25 | 25 |
| Selenium | mcg | 30 | 25 |
| Chromium | mcg | 30 | 40 |
| Molybdenum | mcg | 30 | 30 |

In certain embodiments disclosed in Table 1A, the nutritional composition includes Water, Corn syrup, Sucrose, Milk Protein Concentrate, Sodium Caseinate, Canola Oil, Corn Oil, Fructooligosaccharides, Soy Protein Isolate, Calcium Beta-Hydroxy-Beta-Methylbutyrate, Whey Protein Concentrate, Potassium Citrate, Natural and Artificial Flavors, Potassium Phosphate, Lecithin, Cellulose Gel, Magnesium Hydroxide, Calcium Carbonate, Ascorbic Acid, Calcium Phosphate, Choline Chloride, Sodium Chloride, Sodium Phosphate, Potassium Hydroxide, Zinc Sulfate, Cellulose Gum, L-Carnitine, Carrageenan, dl-Alpha-Tocopherol Acetate, Dextrose, Ferrous Sulfate, Maltodextrin, Niacinamide, Gellan Gum, Calcium Pantothenate, Citric Acid, Cupric Sulfate, Manganese Sulfate, Chromium Chloride, Thiamine Chloride Hydrochloride, Coconut Oil, Vitamin A Palmitate Pyridoxine Hydrochloride, Riboflavin, Folic Acid, Biotin, Sodium Selenate, Sodium Molybdate, Potassium Iodide, Phylloquinone, Cyanocobalamin, and Vitamin D3.

TABLE 1B

| | | Vanilla |
|---|---|---|
| | | Nutritional |
| | | Composition |
| | UNIT | per 8 fl oz |
| Energy EU | kcal | 350 |
| Protein | g | 13 |
| Fat | g | 11 |
| Linoleic acid | g | 0.65 |
| Carbohydrate | g | 51 |
| Fructooligosaccharide | g | 3 |
| Sugar | g | 20 |
| Ca-HMB | g | 1.5 |
| VITAMINS | | |
| Vitamin A (Palmitate) | IU | 1250 |
| Vitamin A (B- | IU | 0 |
| Carotene) | | |
| Vitamin D₃ | IU | 160 |
| Vitamin E | IU | 9 |
| Vitamin K₁ | mcg | 20 |
| Vitamin C | mg | 36 |
| Folic Acid | mcg | 100 |
| Vitamin B₁ | mg | 0.38 |
| Vitamin B₂ | mg | 0.43 |
| Vitamin B₆ | mg | 0.5 |
| Vitamin B₁₂ | mcg | 1.5 |
| Niacin | mg | 5 |
| Pantothenate | mg | 2.5 |
| Biotin | mcg | 75 |
| L-carnitine | mg | 45 |
| Choline | mg | 83 |
| MINERALS | | |
| Sodium | mg | 240 |
| Potassium | mg | 560 |
| Chloride | mg | 68 |
| Calcium | mg | 350 |
| Phosphorus | mg | 350 |
| Magnesium | mg | 100 |
| Iron | mg | 4.5 |
| Zinc | mg | 3.8 |
| Manganese | mg | 1.2 |
| Copper | mg | 0.50 |
| Iodine | mcg | 38 |
| Selenium | mcg | 21 |
| Chromium | mcg | 30 |
| Molybdenum | mcg | 45 |

In a certain embodiment disclosed in Table 1B, the nutritional composition includes Water, Corn Maltodextrin, Sugar, Canola Oil, Sodium Caseinate, Milk Protein Concentrate, Corn Oil, Short-chain Fructooligosaccharides, Soy Protein Isolate, Potassium Citrate, Calcium Beta-Hydroxy-Beta-Methylbutyrate, Whey Protein Concentrate, Natural and Artificial Flavors, Magnesium Phosphate, Soy Lecithin, Sodium Phosphate, Potassium Phosphate, Choline Chloride, Ascorbic Acid, Calcium Carbonate, Potassium Chloride, L-Carnitine, Carrageenan, Ferrous Sulfate, dl-Alpha-Tocopherol Acetate, Zinc Sulfate, Gellan Gum, Niacinamide, Manganese Sulfate, Calcium Pantothenate, Cupric Sulfate, Vitamin A Palmitate, Thiamine Chloride Hydrochloride, Pyridoxine Hydrochloride, Riboflavin, Folic Acid, Chromium Chloride, Biotin, Sodium Molybdate, Sodium Selenate, Potassium Iodide, Phylloquinone, Vitamin D3, Cyanocobalamin.

The following example is included for purposes of illustration and is not intended to limit the scope of the invention.

### EXAMPLE

### Effects of Consuming a High-Calorie Beverage Supplement Including HMB on Post Discharge Incidents in Older Hospitalized Adults

The study objectives are to evaluate the effects of consuming a high-calorie complete and balanced ready to drink flavored beverage supplement providing additional protein, and the leucine metabolite, beta-hydroxy-beta-methylbutyrate, on post discharge incidence of non-elective re-admission and morbid events and medical care consumption in older hospitalized adults.

More specifically, it is the purpose of this study to examine the consumption of two servings of a high-calorie ready to drink flavored beverage containing a total of 700 kcal, 40 g of protein, and 3 g of calcium HMB per day on re-admission rate, functional and nutritional status, morbid events, post-discharge medical care consumption, and quality of life in hospitalized older patients.

### Overall Study Design and Plan: Description

This is a prospective, randomized, double blinded, controlled, two-group parallel design intervention trial. An estimated 652 total elderly eligible patients (≧65 years of age) hospitalized as a result of heart failure, acute myocardial infarction, pneumonia, or chronic obstructive pulmonary disease will be enrolled within 48 hours of admission. Eligible subjects will be stratified based on reason for admission (heart failure, acute myocardial infarction, pneumonia, or chronic obstructive pulmonary disease), gender, and nutritional status and then randomized into two groups (experimental or control). The experimental group (n=326) will receive standard hospital meals and snacks with the experimental ready to drink flavored beverage with the Abbott Nutritionals HMB nutritional composition AN 777, two servings/day. The control group (n=326) will receive standard hospital meals and snacks with the ready to drink control flavored beverage, two servings per day. Throughout the study, both groups will be encouraged to consume the supplement and adequate dietary intake.

### Inpatient Hospital Phase

Study assessments will be conducted during the Inpatient Hospital Phase through 90 days Post Hospital Discharge. Following informed consent, major assessments at Screening (Day −1)/Baseline Day (Day 0) include eligibility criteria, medical history, demographics, blood draw, anthropometrics, severity of illness, functional and nutritional status, and gastrointestinal related adverse events, clinically significant lab values, and serious adverse events. Daily assessments during hospitalization include medications/dietary supplement/study product intake, adverse health events and serious adverse events. At hospital discharge, assessments/procedures include anthropometrics, functional status, medications/dietary supplement/study product intake, documentation of discharge destination or services provided at home (e.g. nursing home, long-term care facility, extended care facility, long term acute care center/hospital, home, home with home care services, rehabilitation center, etc), length of hospital stay (LOS), EQ-5D, SF-36, MMSE, provision of study product instructions and intake records, distribution of study products, and collection of gastrointestinal related adverse events, clinically significant laboratory values, and serious adverse health events.

For all subjects, inpatient meals (with snacks as appropriate) will be provided as usual. Subjects will receive two servings of experimental or control study product each day. The daily volume of study product consumed throughout the study will be assessed on Daily Product Intake Forms.

### Post-Discharge Follow-Up Phase

During the 90-day post-discharge period, the study subjects will be asked to continue to supplement their daily intake with two servings of the experimental ready to drink flavored beverage AN 777 or ready to drink control flavored beverage per day. Study product will be shipped to subjects monthly. At day 30, 60 and 90/Exit visits, assessments/procedures will include anthropometrics, functional and nutritional status, medications/dietary supplements, study product intake, quality of life, morbid events, medical care consumptions, incidence of hospital re-admission, and adverse health events. Blood draw will be at 30 and 60 day visits only. Additional contact via home visit or telephone will be performed weekly to collect morbid events, medical care consumption and maintain study interest, and study product intake.

### Selection of Study Population

The study population will consist of older adults admitted to the hospital for heart failure, acute myocardial infarction, chronic obstructive pulmonary disease, or pneumonia. Subjects eligible for study participation will satisfy the following criteria.

Subjects will be eligible for the study if they meet all of the following inclusion criteria: (1) Subject (male or female) is ≧65 years of age. (2) Subject recently (within 72 hours) admitted to hospital with a primary diagnosis of heart failure, acute myocardial infarction, pneumonia, or chronic obstructive pulmonary disease. (3) Subject has a Subjective Global Assessment rating of B or C at screening. (4) Subject has anticipated length of hospital stay ≧3 days and ≦12 days and is expected to consume ≧2 of the 350 kcal servings of study product while in hospital. (5) Subject is able to consume foods and beverages orally. (6) Subject was functionally ambulatory during the 30 days prior to admission.

### Treatments

Upon fulfilling the eligibility criteria and receiving consent of the subject and HIPAA (or other applicable privacy regulation) authorization prior to any study participation, treatments will be assigned using a prospective computer generated randomization plan. Subjects will be randomly assigned to the experimental or control study product as listed in Table 2:

TABLE 2
Treatments Administered

| | | | Administration |
|---|---|---|---|
| Study Product | Description | Route | Schedule |
| Control Study | Ready to Drink Control | Oral | Two servings/day |
| Product | Flavored Beverage | | |
| Experimental | Ready to Drink Flavored | Oral | Two servings/day |
| Study Product | Beverage with HMB | | |

### Identity of Investigational Product(s)

The approximate compositions of the study products and ingredient listings are shown herein. The experimental study product is a chocolate or vanilla flavored ready to drink beverage having a formulation corresponding to the respective chocolate and vanilla formulations shown in Table 1A. The experimental study product has 350 kcal with 20 g protein, 11 g fat, and 1.5 g calcium HMB (where the calcium HMB ingredient is supplied by Metabolic Technologies, Inc., Ames, Iowa, USA). HMB ingredient has US Generally Regarded As Safe (GRAS) status. The control study product is a ready to drink liquid (Abbott Nutrition, Columbus, Ohio, USA). The control study product has a total calorie content of 48 kcal.

### Method of Assigning Subjects to Treatment

The randomization will be stratified by condition (heart failure, acute myocardial infarction, pneumonia, or chronic obstructive pulmonary disease), gender, and nutritional status by Subjective Global Assessment (malnourished [B or C rating]). To maintain best possible balance within each condition, gender and nutritional status category at a site and within the study overall, a centralized randomization system will be used (Perceptive Informatics).

### Selection and Timing of Levels of Test Product for Each Subject

The experimental study product contains 350 kcal, 20 g protein, and 1.5 g calcium HMB per serving. Two servings per day contain 700 kcal, 40 g protein, and 3 g calcium HMB. Although not statistically different, three grams of HMB showed less loss of lean body mass in a study of older (70±1 yr) men and women during 10 days voluntary bed rest. The control study product contains 48 kcal and 10 mg Vitamin C.

### Treatment Compliance

Product compliance during inpatient hospitalization will be closely monitored daily by study staff. Compliance during the post-discharge phase will be assessed on monthly follow-up visits and at the final 90 day post-discharge study visit/Exit by evaluation of product diaries and return of unused product. Telephone or home visits will be conducted weekly to encourage compliance to product and dietary intake or problem-solve any study issues.

### Discussion and Justification of Study Design

Choice of Study Design and Control Groups: A parallel design was chosen because a cross over design is inappropriate for this study. Subjects with heart failure, acute myocardial infarction, pneumonia, or chronic obstructive pulmonary disease are hospitalized for a short duration, i.e. approximately 6 days on average. It is unlikely that subjects will return to their original baseline following a washout period due to bed rest related loss of lean mass within such a short period.

### Measurement of Results

Efficacy Variable(s) include: (1) Incidence of non-elective all-cause re-admission 30 and 60 days post discharge; (2) Length of stay; and (3) Activities of Daily Living

Supportive Variable(s) include: (1) Study product intake; (2) Weight (% and absolute change) and BMI at hospital discharge, 30, 60 and 90 days post-discharge; and (3) Distribution of nutritional status (Subjective Global Assessment) at hospital discharge, 30, 60 and 90 days post-discharge.

Supportive Variables also include Blood values at baseline, 30 and 60 days post-discharge (total protein, albumin, prealbumin, hemoglobin, hematocrit, red blood cells, white blood cells with differentials [percent and absolute values for lymphocytes, monocytes, neutrophils, eosinophils, basophils], platelet count, glucose, blood urea nitrogen, creatinine, uric acid, sodium, potassium, chloride, calcium, magnesium, phosphorus, total cholesterol, triglycerides, 25-hydroxy vitamin D, parathyroid hormone, and C-reactive protein).

Exploratory Variable(s) include: (1) Quality of Life (EQ-5D and SF-36) at discharge and 30, 60 and 90 days post-discharge; (2) Mini-Mental State Exam at discharge and 30, 60 and 90 days post-discharge; (3) Grip strength (% and absolute change) at discharge, 30, 60, 90 days post-discharge; and (4) Care Transitions Measure®.

Another Exploratory Variable is Factors for Health Economic Outcomes. This includes Discharge destination/service, Post-discharge morbid events (e.g., number of falls, respiratory infections, pneumonia, urinary tract infections, influenza, pressure ulcers), and Post-discharge medical care consumption (general practitioner visits, home care, nursing care, specialist visits, urgent care visits, emergency ward visits, and rehabilitation visits).

Demographic, Anthropometric and Baseline Variable(s) include: (1) Demographics (including age, gender, ethnicity, race, marital status, household size [number of adults and children living in the household], household income, employment status [occupation if employed], health insurance type, education, zip code); and (2) Anthropometrics including Height at screening and Weight and body mass index.

Other Demographic, Anthropometric and Baseline Variable(s) include Distribution of nutritional status (Subjective Global Assessment), Distribution of nutritional status (Malnutrition Screening Tool (MST) at screening, Number of hospital admissions in previous 30 days and 6 months per patient report, Duration of illness or injury prior to hospitalization per patient report, and Severity of illness upon admission.

Subjective Global Assessment (SGA): Subjective Global Assessment will be performed at screening and used for randomization. It is a widely used and practical assessment tool of malnutrition, recommended by the American Society of Parenteral and Enteral Nutrition, providing a classification of malnutrition based on both medical history and clinician observations. Subjective Global Assessment is simple to administer and is widely used in research settings to document and classify patients into one of three categories of nutrition status: well-nourished (A), mildly to moderately (B) or severely (C) malnourished.

Body Weight: Body weight (BW) will be measured using a certified scale and will be calibrated according to manufacturer's instructions.

Height: Height (HT) will be measured without shoes using standard adult height gauge. If a patient is unable to stand up, height will be measured using age and knee height (KH). Height for males=[2.02×knee height]−[0.04×age in years]+64.19; Height for females=[1.83×knee height]−[0.24×age in years]+84.88. Knee height is measured in centimeters with patient in a recumbent position.

Body mass index (BMI): Body mass index (BMI) is a measure for assessing overweight and obesity based on height and weight that applies to both adult men and women. BMI will be calculated by AN at all visits but screening visit. BMI=Weight (kg)/Height (m)².

Handgrip strength (Isometric): Handgrip strength will be used as an indicator of upper body muscle strength. This assessment has been positively correlated with lower-body strength and performance in elderly individuals. Handgrip strength will be measured with a hand-held dynamometer using the subject's dominant arm. The hand-held dynamometer will be standardized and adjusted to the second joint of the finger just below the handle, with the arm adducted at their side and a 90-degree flexion at the elbow. Participants will be asked to squeeze the dynamometer hand as forcefully as possible for three to five seconds. The force will be measured in kilograms. The subject will do three trials with the average used as the final strength value.

Blood tests: Blood will be drawn from a superficial vein for analysis of blood chemistry indicators of health status and inflammatory disease at screening and throughout the study. These include total protein, albumin, prealbumin, hemoglobin, hematocrit, red blood cells, white blood cells with differentials (percent and absolute values for lymphocytes, monocytes, neutrophils, eosinophils, basophils), platelet count, glucose, blood urea nitrogen, creatinine, uric acid, sodium, potassium, chloride, calcium, magnesium, phosphorus, total cholesterol, triglycerides, 25-hydroxy vitamin D, parathyroid hormone, and C-reactive protein and analyzed by a central laboratory. HbA1c, bilirubin, albumin, INR and creatinine will be assessed at screening blood draw and analyzed by hospital laboratory.

Medical Care Consumption: Subjects will report their consumption of general practitioner visits, home care, nursing care, specialist visits, urgent care visits, emergency ward visits, and rehabilitation visits they experienced since the last visit. An outside consulting firm may be used for pharmacoeconomic analyses of these data.

Morbid Events: Subjects will report any incidence of falls, respiratory infections, pneumonia, urinary tract infections, influenza, and pressure ulcers they experienced since the last visit. If an event meets the definition of a serious adverse health event then it must be documented on an AHE Report form. An outside consulting firm may be used for pharmacoeconomic analyses of these data.

Quality of Life: Quality of life will be assessed by two measures, the EQ-5D and SF-36. The EQ-5D is a generic, single index measure of health status developed by a collaborative research network representing experts from countries around the world. Average scores, standardized by age/sex, for both the weighted index and the self-assessed visual analog scale (VAS), are available for the general population and for key population subgroups. EQ-5D is self-administered, reliable and valid and is available in more than 20 languages. This quality of life measure has been used in many clinical trials (Kind 1998, 2002). EQ-5D has five questions based on the areas of mobility, self care, usual activities, pain/discomfort and anxiety/depression and will be used to measure quality of life during the post-discharge phase. Each area has three possible answers, where the first answer of the group is given a value of 1, the second a value of 2 and the third a value of 3, for a total score ranging from 5-15. Health status will also be determined. The Short Form Health Survey is a survey of patient health which measures health status. It is commonly used in health economic studies as a variable in the quality-adjusted life year calculation to determine the cost-effectiveness of a health treatment. The original SF-36 came out from the Medical Outcome Study, MOS, developed by the Research and Development (RAND) Corporation.

Severity of Illness Score: The Charlson Comorbidity Index (CCl) was developed in 1987 based on one-year mortality data from internal medicine patients admitted to a single New York hospital. The CCl is the most extensively studied comorbidity index. The 19 diseases included in the index have been selected and weighted on the basis of the strength of their association with mortality and has been adapted for use with ICD-9 databases. Four out of six comparisons with other indices of comorbidity supported concurrent validity. Predictive validity was confirmed by finding many significant relationships of the Charlson index with various criterion outcomes such as mortality, disability, re-admissions and length of stay. It was initially validated in a cohort of breast cancer patients, has been validated in many cancer types, and is frequently used in oncology drug trials. The reviews of the CCI suggest it is a valid and reliable method to measure comorbidity that can be used in clinical research.

Care Transitions Measure® (CTM-3): The CTM is a patient-centered measure that assesses the extent to which hospital staff accomplished essential care processes. A questionnaire is designed for patients discharged from general acute care hospitals. No proxies are permitted to respond instead of the patient. Someone other than the person who received care is permitted to read the questions to the respondent and/or record the responses. Patients who did not stay at least one night in the hospital may not respond.

Mini-Mental State Exam: The Mini-Mental State Exam is a brief, quantitative measure of cognitive status in adults. It can be used to screen for cognitive impairment, to estimate the severity of cognitive impairment at a given point in time, to follow the course of cognitive changes in an individual over time, and to document an individual's response to treatment. The maximum score on MMSE is 30. A score of 23 or less is indicative of cognitive impairment.

### Suitability of Subject Population

This study is designed to address hospital re-admission in older adults who were hospitalized for heart failure, acute myocardial infarction, pneumonia, or chronic obstructive pulmonary disease. Men and women greater than or equal to (≧) 65 years of age will be eligible. A potential subject will be excluded if they exhibit kidney disease, uncontrolled hypertension, liver impairment or failure and diabetes or any other serious medical illness as diagnosed by the study physician.

### Adverse Health Event

An adverse health event (AHE) is any untoward medical occurrence in a clinical investigation subject which does not necessarily have a causal relationship with a study product. An AHE can therefore be any unfavorable and unintended sign (including abnormal laboratory findings deemed to be clinically significant by the study physician), symptoms, or disease temporally associated with the study, whether or not related to the study product.

Any worsening of a pre-existing condition or illness is considered an AHE. An elective surgery/procedure scheduled to occur during a study will not be considered an AHE if the surgery/procedure is being performed for a pre-existing condition and was pre-planned prior to study entry. However, if the pre-existing condition deteriorates unexpectedly during the trial (e.g. surgery has to be performed earlier than planned), then the deterioration of the condition for which the elective surgery/procedure is being done will be considered an AHE.

### Serious Adverse Health Event

A serious adverse health event (SAHE) is any untoward medical occurrence that results in death, is life-threatening, requires inpatient hospitalization or prolongation of existing hospitalization, results in persistent or significant disability or incapacity, or a congenital anomaly or birth defect. Other important medical events requiring medical or surgical intervention to prevent serious outcome would also be considered SAHEs.

Definitions of events are listed below:

Death: An event that results in the death of a subject.

Life Threatening: An event, which, in the opinion of the physician, would have resulted in immediate fatality if medical intervention had not been taken. This does not include an event that would have been fatal if it had occurred in a more severe form.

Hospitalization: An event that results in an admission to the hospital for any length of time. This does not include an emergency room visit or an admission to an outpatient facility.

Prolongation of Hospitalization: An event that occurs while the study subject is hospitalized and prolongs the subject's hospital stay.

Persistent or Significant: An event that results in a condition that substantially Disability or Incapacity: interferes with the activities of daily living of a subject. “Disability” is not intended to include experiences of relatively minor medical significance such as headache, vomiting or accidental trauma (e.g. sprained ankle)

Congenital Anomaly: An anomaly detected at or after birth or any anomaly that results in fetal loss.

Important Medical Event Requiring Medical or Surgical Intervention to Prevent Serious Outcome: An important medical event may not be immediately life-threatening or result in death or hospitalization but, based on medical judgment, may jeopardize the subject and may require medical or surgical intervention to prevent any of the outcomes listed above.

### Severity

Adverse health events, but not SAHEs, will be categorized into three different levels of severity: mild, moderate and severe. These categories are defined as follows:

Mild: Transient and easily tolerated.

Moderate: Results in a modification or interruption of the subject's usual activities or care and may require discontinuation of the study product.

Severe:Results in considerable interference with the subject's usual activities or care and may require discontinuation of study product.

### Relationship to Study Product

A physician, who is an investigator or subinvestigator for the study, will assess the relationship of the AHE or SAHE to the study product using the following definitions:

Probable: An AHE or SAHE has a strong temporal relationship to the study product or recurs on re-challenge and another etiology is unlikely or significantly less likely.

Possible: An AHE or SAHE has a strong temporal relationship to the study product and an alternative etiology is equally or less likely compared to the potential relationship to study product.

Probably Not: An AHE or SAHE has little or no temporal relationship to the study product and/or a more likely alternative etiology exists.

Not Related: An AHE or SAHE is due to an underlying or concurrent illness and is not related to the study product.

If the investigator's opinion is that the event is possibly, probably not, or not related to the study product, an alternative etiology must be provided for the AHE.

### Statistical and Analytical Plans

This is a prospective, randomized, double-blinded, controlled, two-group, parallel design, multicenter intervention trial to evaluate the effects of consuming a high-calorie complete and balanced oral nutritional supplement providing additional protein, and calcium HMB on post discharge incidence of re-admission, morbid events and medical care consumption in older hospitalized adults.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing possible mechanisms through with the compounds of the invention are effective, the inventors are not bound by theories described herein. The invention is not limited to the exact details shown and described, for variations obvious to one skilled in the art will be included within the invention defined by the claims.

1-20. (canceled)

21. A method of decreasing a risk of an adverse health event during or subsequent to a hospitalization of an adult or elderly patient, comprising administering an effective amount of β-hydroxy-β-methylbutyrate to the patient during the hospitalization.

22. The method of claim 21, wherein the β-hydroxy-β-methylbutyrate is administered orally.

23. The method of claim 22, wherein the β-hydroxy-β-methylbutyrate is administered as part of a nutritional composition.

24. The method of claim 23, wherein the nutritional composition further comprises at least one source of protein, at least one source of carbohydrate, and at least one source of fat.

25. The method of claim 23, wherein the nutritional composition comprises about 150-1000 kilocalories per serving, and
wherein the nutritional composition is one of a liquid, a powder suitable for reconstitution to a liquid, or a bar.

26. The method of claim 23, wherein the nutritional composition comprises about 350-500 kilocalories per serving, and
wherein the nutritional composition comprises at least about 10 grams of protein per serving.

27. The method of claim 21, wherein the β-hydroxy-β-methylbutyrate comprises calcium β-hydroxy-β-methy lbutyrate.

28. The method of claim 21, wherein the effective amount of β-hydroxy-β-methylbutyrate is from about 0.1 g/day to about 10 g/day.

29. The method of claim 21, wherein the effective amount of β-hydroxy-β-methylbutyrate is from about 0.5 g/day to about 5 g/day.

30. The method of claim 21, wherein the β-hydroxy-β-methylbutyrate is administered at least once per day.

31. The method of claim 21, wherein the β-hydroxy-β-methylbutyrate is also administered to the patient after the hospitalization has ended.

32. The method of claim 31, wherein the β-hydroxy-β-methylbutyrate is administered to the patient during and up to about 1 year after the hospitalization has ended.

33. The method of claim 21, wherein the patient is at least 65 years old.

34. The method of claim 21, wherein the patient was malnourished immediately prior to administration of the β-hydroxy-β-methylbutyrate.

35. The method of claim 21, wherein the adverse health event comprises death, incapacity, or a health event that is life threatening.

36. The method of claim 21, wherein the adverse health event is selected from the group consisting of an injurious fall, a respiratory infection, a urinary tract infection, pressure ulcers, and acute cardiac events.

37. The method of claim 21, wherein the adverse health event is selected from the group consisting of pneumonia, influenza, and thrombosis.

38. The method of claim 21, wherein the adverse health event requires an increase in a duration of the hospitalization in comparison to an expected duration of the hospitalization.

39. The method of claim 21, wherein the adverse health event requires medical care after the hospitalization has ended, the medical care selected from the group consisting of a visit to a health practitioner, a home visit by a health practitioner, entry into a nursing home, rehabilitation therapy, and rehospitalization.

40. The method of claim 21, wherein the patient has sarcopenia.

41. A method of decreasing a risk of an adverse health event during or subsequent to a hospitalization of an elderly patient, the method comprising administering from about 1 g/day to about 10 of β-hydroxy-β-methylbutyrate in a liquid nutritional supplement to the patient, wherein the administration occurs at least once per day during both the hospitalization of the patient and up to about one year after the hospitalization has ended.

42. The method of claim 41, wherein the adverse health event is selected from the group consisting of an injurious fall, a respiratory infection, a urinary tract infection, pressure ulcers, and acute cardiac events.

43. The method of claim 41, wherein the adverse health event is selected from the group consisting of pneumonia, influenza, and thrombosis.

44. The method of claim 41, wherein the adverse health event requires an increase in a duration of the hospitalization in comparison to an expected duration of the hospitalization.

45. The method of claim 41, wherein the patient has sarcopenia.
